(19) United States
(12) Patent Application Publication
(10) Pub. No.: US 2013/0303781 A1
(43) Pub. Date: Nov. 14, 2013

(54) PROCESS FOR PREPARATION OF TRICLABENDAZOLE

(71) Applicants: Ramkrishna Appaji Rane, Marg (IN); Sudhakar Verma, Thane (IN); Thangavel Arulmoli, Karnataka (IN)

(72) Inventors: Ramkrishna Appaji Rane, Marg (IN); Sudhakar Verma, Thane (IN); Thangavel Arulmoli, Karnataka (IN)

(73) Assignee: SEQUENT SCIENTIFIC LIMITED, Mangalore, Karnataka (IN)

(21) Appl. No.: 13/989,147

(22) PCT Filed: Nov. 23, 2011
(86) PCT No.: PCT/IN2011/000810

§ 371 Date: Jul. 31, 2013

(30) Foreign Application Priority Data
Nov. 24, 2010 (IN) 3538/CHE/2010

(51) Int. Cl. C07D 235/28

(52) U.S. Cl. CPC C07D 235/28
USPC 548/307.1

(57) ABSTRACT

The present invention discloses a method for preparing Triclabendazole comprising condensing N-(4,5-dichloro-2-ni-trophenyl)acetamide with 2,3-dichlorophenol to obtain 4-chloro-5(2,3-dichlorophenoxy)-2-nitrophenyl acetamide and it to obtain 4-chloro-5(2,3-dichlorophenoxy)-2-nitroaniline; reducing 4-chloro-5(2,3-dichlorophenoxy)-2-nitroaniline in presence of Raney nickel to obtain 4-chloro-5-(2,3-dichlorophenoxy)benzene-1,2-diamine of; cyclising 4-chloro-5-(2,3-dichlorophenoxy)benzene-1,2-diamine in presence of carbondisulfide to obtain 6-chloro-5-(2,3-dichlorophenoxy)-1H-benzimidazole-2-thiol; methylating 6-chloro-5-(2,3-dichlorophenoxy)-1H-benzimidazole-2-thiol using a methylating agent to obtain triclabendazole methanesulfonate salt; converting triclabendazole methanesulfonate salt to hydrochloride salt of Triclabendazole and hydrolysing it to obtain Triclabendazole.

### FIELD OF INVENTION

The present invention relates to a novel, cost-effective process for preparation of Triclabendazole.

### BACKGROUND OF THE INVENTION

Triclabendazole, chemically known as 5-chloro-6-(2,3-dichlorophenoxy)-2-(methylthio)-1H-benzimidazole represented by formula I,

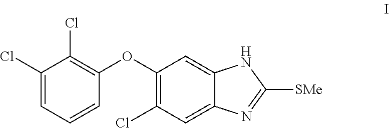 [SMILES: CSc1nc2cc(Cl)c(Oc3cccc(Cl)c3Cl)cc2[nH]1]

is a halogenated benzimidazole compound that possesses high activity against immature and adult stages of the liver fluke, Faciola hepatica. The intensive use of Triclabendazole in endemic areas of facioliasis has resulted in the development of liver flukes resistant to this compound. U.S. Pat. No. 4,197,307 discloses the process for the preparation of Triclabendazole, wherein 4-chloro-5-(2,3-dichlorophenoxy)-1,2-benzenediamine is reacted with carbondisulfide to give cyclic benzimidazole thione, which is further subjected to alkylation reaction with dimethyl sulfate to give Triclabendazole.

Chinese patent 101555231 describes a process for the preparation of Triclabendazole by hydrolysing N-(4,5-dichloro-2-nitrophenyl)acetamide of formula VII to 4,5-dichloro-2-nitroaniline of formula VIII and condensing it with 2,3-dichlorophenol of formula VI in presence of a phase transfer catalyst to obtain 4-chloro-5(2,3-dichlorophenoxy)-2-nitroaniline of formula IV, which is further reduced in presence of Iron to obtain 4-chloro-5-(2,3-dichlorophenoxy)benzene-1,2-diamine of formula III. The obtained diamine of formula III is cyclised in presence of carbondisulfide to obtain 6-chloro-5-(2,3-dichlorophenoxy)-1H-benzimidazole-2-thiol of formula II. The compound of formula II is methylated using dimethyl sulphate to obtain Triclabendazole of formula I. The process disclosed in this patent is illustrated in scheme 1 below:

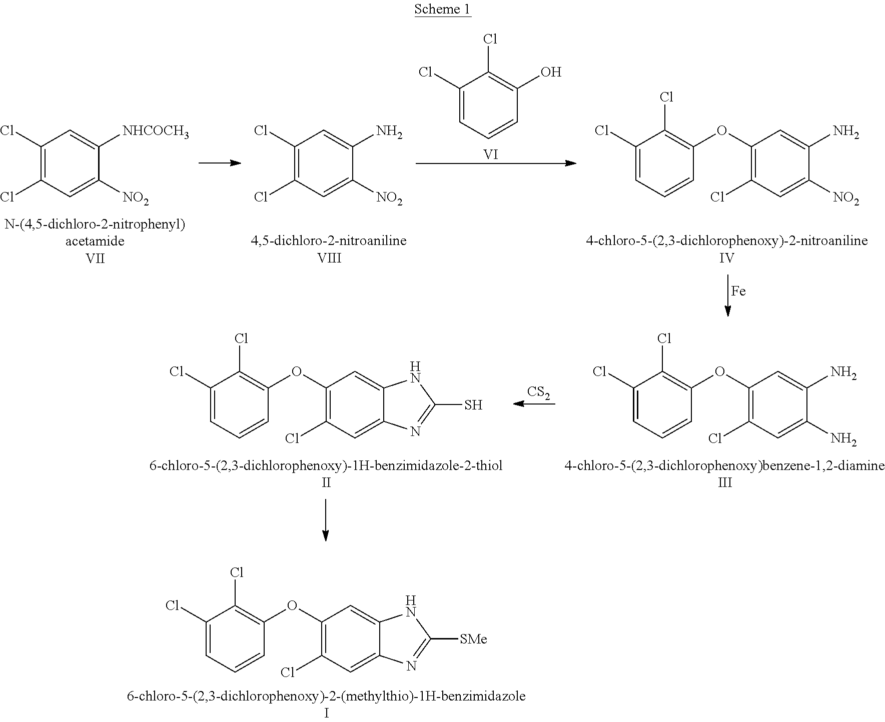

However, the above prior art process is not preferred at a commercial scale because the hydrolysis of N-(4,5-dichloro-2-nitrophenyl)acetamide of formula VII is carried out before condensation with 2,3-dichlorophenol of formula VI, which is labile to formation of impurities and moreover the condensation is carried out in the presence of a phase transfer catalyst. Further, Iron is used as a catalyst for reduction which is not environment friendly and involves tedious work-up. The final compound Triclabendazole is directly obtained by the methylating the compound of formula II using dimethylsulfate. The purity of thus obtained Triclabendazole is not high.

Thus it is highly desirable to develop a process which overcomes most of the prior art drawbacks. The present inventors have developed a process for the preparation of Triclabendazole, which is environment friendly, technologically safe, simple and cost effective

### SUMMARY OF THE INVENTION

The principal aspect of the present invention is to provide a process for the preparation of Triclabendazole comprising:
a) condensing N-(4,5-dichloro-2-nitrophenyl)acetamide of formula VII with 2,3-dichlorophenol of formula VI to obtain 4-chloro-5(2,3-dichlorophenoxy)-2-nitrophenyl acetamide of formula V;
b) hydrolysing 4-chloro-5(2,3-dichlorophenoxy)-2-nitrophenyl acetamide of formula V to obtain 4-chloro-5(2,3-dichlorophenoxy)-2-nitroaniline of formula IV;
c) reducing 4-chloro-5(2,3-dichlorophenoxy)-2-nitroaniline of formula IV in presence of Raney nickel to obtain 4-chloro-5-(2,3-dichlorophenoxy)benzene-1,2-diamine of formula III;
d) cyclising 4-chloro-5-(2,3-dichlorophenoxy)benzene-1,2-diamine of formula III in presence of carbondisulfide to obtain 6-chloro-5-(2,3-dichlorophenoxy)-1H-benzimidazole-2-thiol of formula II;
e) methylating 6-chloro-5-(2,3-dichlorophenoxy)-1H-benzimidazole-2-thiol of formula II using a methylating agent to obtain Triclabendazole methanesulfonate salt of formula IX;
f) converting Triclabendazole methanesulfonate salt of formula IX to hydrochloride salt of Triclabendazole of formula X; and
g) hydrolysing Triclabendazole hydrochloride of formula X to obtain Triclabendazole of formula I.

The process of the present invention is illustrated in scheme 2 below:

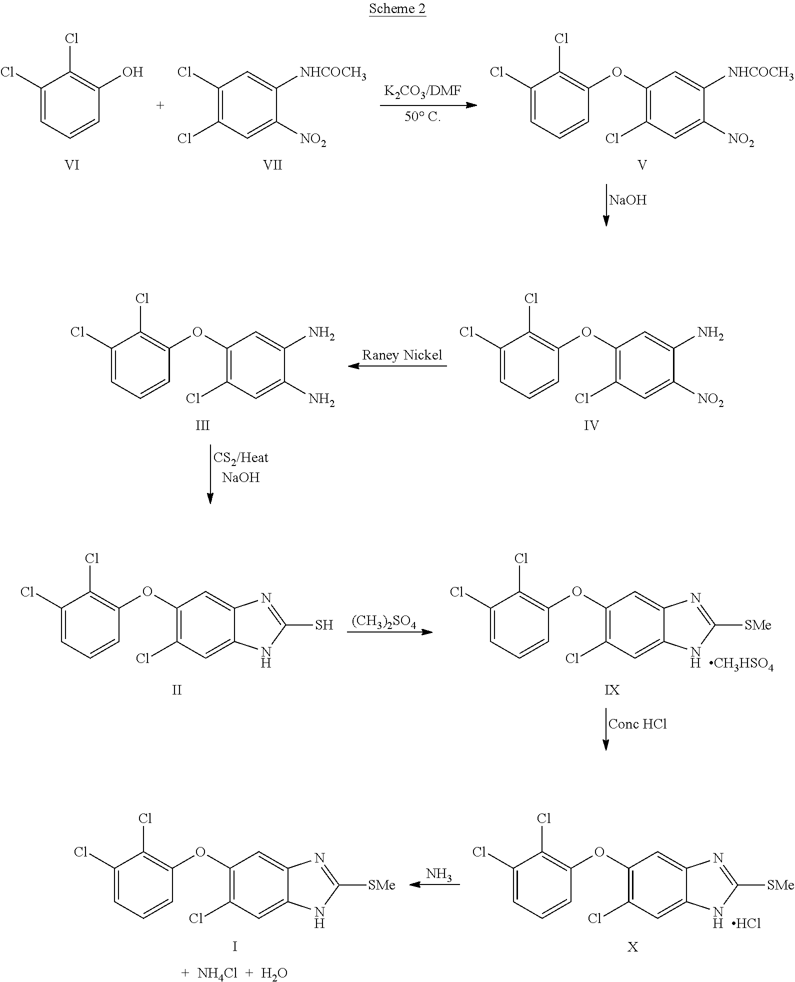

### DETAIL DESCRIPTION OF THE INVENTION

Accordingly in an embodiment of the invention, the condensation and hydrolysis in step a) and b) is carried out in-situ in presence of a polar aprotic solvent like dimethylformamide (DMF), DMSO, sulfolane, N-methylpyrrolidinone and alcoholic solvent like methanol at a temperature 30° C. to 100° C. preferably at 50° C. to 90° C. The base used for the condensation and hydrolysis are sodium carbonate, potassium carbonate, or sodium hydroxide or potassium hydroxide. The condensation is preferably carried out in presence of solvent dimethylformamide and base potassium carbonate whereas the hydrolysis is done in presence of sodium hydroxide. In further embodiment no phase transfer catalyst is required for the condensation of the present invention.

In another embodiment of the invention, the reduction in step c) is carried out in an alcoholic solvent like methanol, ethanol, isopropanol, preferably in presence of methanol and Raney nickel and sodium hydroxide. Preferably the reduction is carried out at H₂ pressure of 6 kg and at temperature about 100° C.

In another embodiment the obtained 4-chloro-5(2,3-dichlorophenoxy)-1,2-phenylenediamine in step c is directly cyclized with carbon disulfide in presence of strong base such as caustic lye without isolating followed by acidification with acetic acid.

In another embodiment of the invention, the cyclisation or ring closure in step d) is carried out at a temperature 50° C. to 140° C. in presence of carbondisulfide, a solvent selected from dimethylformamide, methanol, ethanol, or acetonitrile, preferably methanol and in presence of a base like sodium hydroxide.

In another embodiment of the invention, methylation 6-chloro-5-(2,3-dichlorophenoxy)-1H-benzimidazole-2-thiol of formula H in step e) is carried out using a methylating agent like dimethylsulfate in temperature range of 20 to 80° C. preferably 40 to 65° C. and in presence of an alcoholic solvent preferably methanol to obtain Triclabendazole methanesulfonate salt.

In another embodiment the obtained 4-chloro-5(2,3-dichlorophenoxy)-1,2-phenylenediamine in step c is directly cyclized with carbon disulfide in presence of strong base such as caustic lye without isolating followed by acidification with acetic acid.

In yet another embodiment of the invention, Triclabendazole methanesulfonate salt is dissolved in an alcoholic solvent preferably methanol, charcolated and added concentrated hydrochloric acid and cooled to isolate hydrochloride salt of Triclabendazole. Which is further treated with water and ammonia to obtain Triclabendazole.

In yet another embodiment of the invention, 6-chloro-5-(2,3-dichlorophenoxy)-1H-benzimidazole-2-thiol of formula II is methylated using a methylating agent like dimethylsulfate in presence of an alcoholic solvent preferably methanol and a base preferably sodium carbonate in temperature range of 40 to 90° C. preferably 60 to 90° C. to obtain Triclabendazole directly.

In still further embodiment of the invention the obtained triclabendazole is optionally purified by dissolving in toluene at 90-100° C., removing water azeotropically, cooling & charcolyzing, and again adding isopropanol at 90-100° C.

The preferred embodiment of the invention can be illustrated by the below given examples, however it should not be construed to limit the scope of the invention.

### Example 1

### Preparation of 5-chloro-6-(2,3-dichlorophenoxy)-2-(methylthio)-1H-benzimidazole (I)

### (a) Preparation of 4-chloro-5(2,3-dichlorophenoxy)-2-nitroaniline

2,3-dichlorophenol (1 kg) in DMF (1.5 L), 2-nitro 4,5-dichloroacetanilide (1.52 kg), and potassium carbonate were heated into the flask for 12 hrs while maintaining the temperature at 90° C. under vacuum and after that cooled to room temperature. Methanol (2 L), 48% caustic lye (0.3 kg) in 300 mL water were added to it and heated to 50° C. for 4 hrs. Further water (4 L) was added, stirred, filtered and washed with water and with methanol.

### Weight=2 kg.

### (b) Preparation of 4-chloro-5(2,3-dichlorophenoxy)-1,2-phenylenediamine

Raney nickel (10.8 g) was added into a reaction mixture containing 4-chloro-5(2,3-dichlorophenoxy)-2-nitroaniline (900 g), methanol (3.4 L) at RT, caustic lye (2.72 g). Nitrogen was flushed into and charged with hydrogen. The reaction mixture was heated slowly to 100° C. for 12 hrs, cooled to RT and filtered.

### Weight: 819 g

### (c) Preparation of 6-chloro-5(2,3-dichlorophenoxy)-1H-benzimidazole-2-thiol

In the mixture of 4-chloro-5(2,3-dichlorophenoxy)-1,2-phenylenediamine in methanol (800 g) and caustic lye (245 mL), carbondisulfide (259 g) was added slowly and the reaction mass was refluxed for 6 hrs. After completion of the reaction water (2.5 L) and acetic acid was added over a period of 2 hrs at 60° C. Water was added (2.5 litre) again and heated to 90° C. for 2 hrs, filtered and washed with hot water to obtain the title compound.

Weight: 863 g.

### (d) Preparation of 6-chloro-5(2,3-dichlorophenoxy)-2-(methylthio)-1H-benzimidazole

6-chloro-5(2,3-dichlorophenoxy)-1H-benzimidazole-2-thiol(400 kg) was added to methanol (700 L) and heated to 40° C. Dimethyl sulphate was added slowly at 40° C. to it. The reaction mass was heated to 60-65° C. and maintain for 6 hrs. After completion of the reaction the reaction mass was cooled to 15° C., centrifuged the material and washed with 75 L of methanol to obtain wet cake of Triclabendazole methanesulfonate (520-560 kg).

Triclabendazole methanesulfonate (200 g) and methanol (1.2 L) was refluxed, cooled and charcoal was added and refluxed again for 1 hr. The reaction mass was filtered and concentrated hydrochloric acid was added. The precipitate was cooled to RT, stirred for 1 hr, filtered and Triclabendazole hydrochloride was isolated (250 g wet).

The water was added to the above Triclabendazole hydrochloride and ammonia was charged and stirred for 2-3 hrs. The reaction mass was filtered, washed with water and dried to obtain Triclabendazole.

### Weight: 156 g.

### Example 2

### Preparation of 6-chloro-5(2,3-dichlorophenoxy)-2-(methylthio)-1H-benzimidazole

In a RBF methanol (200 mL), 6-chloro-5(2,3-dichlorophenoxy)-1H-benzimidazole-2-thiol ((200 g) and dimethylsulfate (40 g) were heated to 60±2° C. and water (100 mL) was added and stirred for half an hr. Sodium carbonate solution (25 g Na₂CO₃ in 200 mL water) was added slowly and temperature was raised to 60° C. and stirred for 1½ hr. After completion of reaction, the reaction mixture was cooled to 60° C., filtered, washed with water further washed with toluene and dried.

To the above wet crude 6-chloro-5(2,3-dichlorophenoxy)-2-(methylthio)-1H-benzimidazole, toluene (500 mL) was charged and water was removed azeotropically using Dean Stark apparatus. The mixture was heated to 100-112° C. and 5 g charcoal was added, stirred for half an hr at 100-105° C. The reaction mixture was filtered through hyflow bed and washed with fresh toluene. The mother liquor was cooled to 70° C. and isopropanol (7 mL) was added, cooled to room temperature to precipitate, filtered and washed with fresh toluene, dried at 75° C. for 4 hrs to obtain pure Triclabendazole.

Yield of Triclabendazole is 85 gm. (81.7%).

### Example 3

### Purification of Triclabendazole

The wet cake of Triclabendazole was heated to 90-100° C. in toluene (1.92 litre). Water was removed azeotropically. The solution/mixture was cooled charcoal was added, refluxed and filtered. Again the obtained material was heated to 90-100° C., 180 ml of IPA was added, cooled to RT, filtered and dried for 24 hrs at 90-100° C.

Wt: 132 g (1^{st} crop) and 12±1 g (2^{nd} crop)

1. A process for the preparation of Triclabendazole comprising:
a) condensing N-(4,5-dichloro-2-nitrophenyl)acetamide with 2,3-dichlorophenol to obtain 4-chloro-5(2,3-dichlorophenoxy)-2-nitrophenyl acetamide;
b) hydrolysing 4-chloro-5(2,3-dichlorophenoxy)-2-nitrophenyl acetamide to obtain 4-chloro-5(2,3-dichlorophenoxy)-2-nitroaniline of;
c) reducing 4-chloro-5(2,3-dichlorophenoxy)-2-nitroaniline in the presence of Raney nickel to obtain 4-chloro-5-(2,3-dichlorophenoxy)benzene-1,2-diamine;
d) cyclising 4-chloro-5-(2,3-dichlorophenoxy)benzene-1,2-diamine in presence of carbondisulfide to obtain 6-chloro-5-(2,3-dichlorophenoxy)-1H-1-benzimidazole-2-thiol; and
e) methylating 6-chloro-5-(2,3-dichlorophenoxy)-1H-benzimidazole-2-thiol using a methylating agent to obtain Triclabendazole.

2. A process for the preparation of Triclabendazole according to claim 1, wherein the condensation and hydrolysis in step a) and b) is carried out in-situ in presence of solvent selected from the group consisting of dimethylformamide (DMF), DMSO, sulfolane, N-methylpyrrolidinone and methanol at a temperature of between 30° C. and 100° C.

3. A process for the preparation of Triclabendazole according to claim 1, wherein the condensation and hydrolysis in step a) and b) is carried out in-situ in presence of a base selected from the group consisting of sodium carbonate, potassium carbonate, sodium hydroxide and potassium hydroxide.

4. (canceled)

5. A process for the preparation of Triclabendazole according to claim 1, wherein the reduction in step c) is carried out in the presence of an alcoholic solvent and a base.

6. A process for the preparation of Triclabendazole according to claim 1, wherein cyclisation in step d) is carried out in the presence of:
a solvent selected from the group consisting of dimethylformamide, methanol, ethanol, acetonitrile and a mixture thereof; and
a base.

7. A process for the preparation of Triclabendazole according to claim 1, where in methylation of 6-chloro-5-(2,3-dichlorophenoxy)-1H-benzimidazole-2-thiol to obtain Triclabendazole comprises:
i) methylating 6-chloro-5-(2,3-dichlorophenoxy)-1H-benzimidazole-2-thiol using dimethylsulfate as a methylating agent to obtain Triclabendazole methanesulfonate salt;
ii) converting Triclabendazole methanesulfonate salt to a hydrochloride salt of Triclabendazole; and
iii) converting Triclabendazole hydrochloride into Triclabendazole.

8. A process for the preparation of Triclabendazole according to claim 1, where in 6-chloro-5-(2,3-dichlorophenoxy)-1H-benzimidazole-2-thiol is methylated using a methylating agent in the presence of an alcoholic solvent and a base in a temperature range of 40 to 90. C to obtain Triclabendazole.

9. A process for the preparation of Triclabendazole according to claim 1, wherein the methylating agent used is dimethylsulfate.

10. (canceled)

11. A process for the preparation of Triclabendazole according to claim 1, further comprising:
f) purifying the Triclabendazole obtained in step (e) by crystallization from a mixture of toluene and isopropanol.

12. A process for the preparation of Triclabendazole comprising:
a) condensing N-(4,5-dichloro-2-nitrophenyl)acetamide with 2,3-dichlorophenol to obtain 4-chloro-5(2,3-dichlorophenoxy)-2-nitrophenyl acetamide,
said condensing being carried out in the absence of a phase transfer catalyst;
b) hydrolysing 4-chloro-5(2,3-dichlorophenoxy)-2-nitrophenyl acetamide to obtain 4-chloro-5(2,3-dichlorophenoxy)-2-nitroaniline;
c) reducing the nitro group of 4-chloro-5(2,3-dichlorophenoxy)-2-nitroaniline to obtain 4-chloro-5-(2,3-dichlorophenoxy)benzene-1,2-diamine;
d) cyclising 4-chloro-5-(2,3-dichlorophenoxy)benzene-1,2-diamine in presence of carbondisulfide to obtain 6-chloro-5-(2,3-dichlorophenoxy)-1H-benzimidazole-2-thiol; and
e) methylating 6-chloro-5-(2,3-dichlorophenoxy)-1H-benzimidazole-2-thiol using a methylating agent to obtain Triclabendazole.
